(19) United States
(12) Patent Application Publication
(10) Pub. No.: US 2012/0177760 A1
(43) Pub. Date: Jul. 12, 2012

(54) COSMETIC OR DERMOTOLOGICAL COMPOSITION CONTAINING HYDROLYZATES MADE OF PLANT EXTRACTS

(75) Inventor: Michael Popp, Lauf-Heuchling (DE)

(73) Assignee: Bionorica SE, Neumarkt (DE)

(21) Appl. No.: 13/266,645

(22) PCT Filed: Apr. 29, 2010
(86) PCT No.: PCT/EP2010/055875

§ 371 Date: Mar. 19, 2012

(30) Foreign Application Priority Data
Apr. 29, 2009 (DE) 10 2009 019 104.6

(51) Int. Cl. A61K 8/97; A61K 36/515; A61K 36/85; A61K 36/70; A61P 31/04; A61P 17/10; A61P 17/00; A61K 36/185; A61Q 19/00

(52) U.S. Cl. 424/769; 424/725

(57) ABSTRACT

The invention relates to a cosmetic or dermatological composition or a means for body care and beauty care, preferably on a pure plant basis, for topical use, containing at least one plant hydrolyzate, in particular selected from the group Althaeae radix (marshmallow root), Angelica(e) dahurica (dahurian angelica), Angelica(e) sinensis (Chinese angelica), Armoracia rusticana (horseradish), Artemisia scoparia (redstem wormwood), Astragalus membranaceus (var. mongolicus) (Chinese milk vetch), Capsicum sp., Capsicum annuum (pepper), Centaurium erythraea (common centaury), Cistus incanus (hairy rockrose), Echinacea angustifolia (narrow-leaved purple coneflower), Echinacea purpurea (eastern purple coneflower), Equiseti herba (horsetail), Galphimia glauca, Gentiana lutea L. (great yellow gentian), Guaiacum officinalis, Hederae folium, Hedera helicis (ivy), Juglandis folium (walnut leaf), Leonurus japonicus (Chinese motherwort), Levesticum radix, Levisticum officinalis (lovage), Matricariae flos (or Flos chamomillae (chamomile flower)), Melia toosendan (chinaberry fruit), Millefolii herba (yarrow), Olea europaea (olive), Pelargonium sp. (geranium), Phytolacca americana (American pokeweed), Primulae flos, Primulae radix, Primula yeris L. (cowslip), Quercus cortex (oak bark), Rosmarinus officinalis (rosemary), Rumicis herba (sorrel herb), Salix sp. (willow), Salvia miltiorrhiza (red sage), Sambucus nigra L. (black elder), Saposhnikovia divaricata (siler), Scutellaria baicalensis (Baikal skullcap), Siegesbeckia pubescens (Siegesbeckia), Taraxaci herba (dandelion root herb), Thymus L. (thyme), Verbena officinalis L. (vervain), Vitex agnus castus (monk's pepper), Vitis vinifera (common grape vine).

The invention relates to a cosmetic or dermatological composition or an agent for body care and beauty care, preferably on a pure plant basis, for topical use, containing at least one plant hydrolysate, in particular selected from the group Althaeae radix (marshmallow root), Angelica(e) dahurica (dahurian angelica), Angelica(e) sinensis (Chinese angelica), Armoracia rusticana (horseradish), Artemisia scoparia (redstem wormwood), Astragalus membranaceus (var. mongolicus) (Chinese milk vetch), Capsicum sp., Capsicum annuum (pepper), Centaurium erythraea (common centaury), Cistus incanus (hairy rockrose), Echinacea angustifolia (narrow-leaved purple coneflower), Echinacea purpurea (Eastern purple coneflower), Equiseti herba (horsetail), Galphimia glauca, Gentiana lutea L. (great yellow gentian), Guaiacum officinalis, Hederae folium, Hedera helicis (ivy), Juglandis folium (walnut leaf), Leonurus japonicus (Chinese motherwort), Levesticum radix, Levisticum officinalis (lovage), Matricariae fibs (or Flos chamomillae (chamomile flower)), Melia toosendan (chinaberry fruit), Millefolii herba (yarrow), Olea europaea (olive), Pelargonium sp. (geranium), Phytolacca americana (American pokeweed), Primulae flos, Primulae radix, Primula veris L. (cowslip), Quercus cortex (oak bark), Rosmarinus officinalis (rosemary), Rumicis herba (sorrel herb), Salix sp. (willow), Salvia miltiorrhiza (red sage), Sambucus nigra L. (black elder), Saposhnikovia divaricata (siler), Scutellaria baicalensis (Baikal skullcap), Siegesbeckia pubescens (Siegesbeckia), Taraxaci herba (dandelion root herb), Thymus L. (thyme), Verbena officinalis L. (vervain), Vitex agnus castus (monk's pepper), Vitis vinifera (common grapevine).

High demands are to be placed on a modern cosmetic or dermatological composition or an agent for body care and beauty care, preferably on a pure plant basis, for topical use.

As the largest organ of humans, the skin exercises numerous functions important to life. For example, it protects from cold, heat, radiation, the effect of chemical substances, and pathogens. If the skin no longer adequately fulfills these barrier functions, local irritations or also illnesses of the entire body, such as infections, may occur.

The skin, in particular the epidermis, is subjected to external effects to a particular degree as the barrier organ of the human organism. This barrier function is maintained, inter alia, by skin lipids. These epidermal lipids, such as glycosphingolipids, ceramides, sterols and sterol esters, fatty acids, triglycerides, n-alkanes, or various polar lipids are released in the keratinization process.

A balanced ratio of skin lipids and skin moisture is present in an optimum state of the skin. Important properties of the skin are determined by this equilibrium, such as the penetration capability, water binding capability, elasticity, regeneration capability, or the resistance capability to environmental influences and noxious substances of various types. The skin lipids, particularly the surface lipids, are to be ascribed an overriding significance in this case.

The external lipid film of the skin represents a very complexly composed emulsion made of various lipids and the secretions of the sweat glands, such as urea, fatty acids, inorganic salts, and water. The components of the oil phase predominantly originate from the secretions of the sebaceous glands, which contain, inter alia, squalene, cholesterol and cholesterol esters, esters of the wax type, triglycerides, and free fatty acids.

The invention relates to a cosmetic or dermatological composition or an agent for body care and beauty care containing a hydrolysate made of at least one extract of at least one plant material selected from the respective genera, in particular the species

Althaeae radix (marshmallow root), Angelica(e) dahurica (dahurian angelica), Angelica(e) sinensis (Chinese angelica), Armoracia rusticana (horseradish), Artemisia scoparia (redstem wormwood), Astragalus membranaceus (var. mongolicus) (Chinese milk vetch), Capsicum sp., Capsicum annuum (pepper), Centaurium erythraea (common centaury), Cistus incanus (hairy rockrose), Echinacea angustifolia (narrow-leaved purple coneflower), Echinacea purpurea (Eastern purple coneflower), Equiseti herba (horsetail), Galphimia glauca, Gentiana lutea L. (great yellow gentian), Guaiacum officinalis, Hederae folium, Hedera helicis (ivy), Juglandis folium (walnut leaf), Leonurus japonicus (Chinese motherwort), Levesticum radix, Levisticum officinalis (lovage), Matricariae Bps (or Flos chamomillae (chamomile flower)), Melia toosendan (chinaberry fruit), Millefolii herba (yarrow), Olea europaea (olive), Pelargonium sp. (geranium), Phytolacca americana (American pokeweed), Primulae flos, Primulae radix, Primula veris L. (cowslip), Quercus cortex (oak bark), Rosmarinus officinalis (rosemary), Rumicis herba (sorrel herb), Salix sp. (willow), Salvia miltiorrhiza (red sage), Sambucus nigra L. (black elder), Saposhnikovia divaricata (siler), Scutellaria baicalensis (Baikal skullcap), Siegesbeckia pubescens (Siegesbeckia), Taraxaci herba (dandelion root herb), Thymus L. (thyme), Verbena officinalis L. (vervain), Vitex agnus castus (monk's pepper), Vitis vinifera (common grapevine), or a mixture or (sub) combination thereof, and a method for the production and the use thereof.

The mentioned healing plants have already been used as pharmaceuticals, e.g., Sinupret®, Tonsipret®, or Canephron® (registered trademarks of BIONORICA AG).

Sinupret® is a known mixture of five plant drugs, namely Verbena officinalis L. (vervain), Sambucus nigra L. (black elder), Primula veris L. (cowslip), Rumicis herba (sorrel herb), Gentiana lutea L. (great yellow gentian).

Tonsipret® is a known mixture of three plant drugs, namely Guaiacum officinalis, Phytolacca americana (American pokeweed), and Capsicum annuum (pepper).

Canephron® is a known mixture of three plant drugs, namely Centaurium erythraea (common centaury), Levisticum officinalis (lovage; powder of lovage roots), Rosmarinus officinalis (rosemary; powder of rosemary leaves), which is provided from these plant materials. Through a combination of these mentioned healing plants, a composition is obtained, using which an effect sufficient for medical and dermatological purposes can be achieved. The ground crude drugs and ethanol-aqueous extracts or dry extracts produced therefrom (producible by withdrawing the solvent/extracting agent under reduced pressure, for example) of the above-mentioned plants have proven themselves through their healing power, which is exclusively originates from a plant base. The healing plants used in Bionorica® are carefully selected, assayed, and further processed. Bionorica® achieves the uniform quality of the pharmaceuticals through optimally worked out cultivation and harvesting strategies and extremely strict quality control.

Angelica dahurica, Angelica sinensis, Artemisia scoparia, Astragalus membranaceus (var. mongolicus), Leonurus japonicus, Salvia miltiorrhiza, Saposhnikovia divaricata, Scutellaria baicalensis, and Siegesbeckia pubescens are known representatives of traditional Chinese medicine (TCM) and are described for numerous indications.

Furthermore, plant genera and species exist, such as Armoracia rusticana, Capsicum sp., Capsicum annuum, Cistus incanus, Echinacea angustifolia, Echinacea purpurea, Galphimia glauca, Hedera helicis, Melia toosendan, Olea europaea, Pelargonium sp., Phytolacca americana, Primula veris, Salix sp., Thymus L., Vitex agnus castus, and Vitis vinifera, to each of which a pharmaceutical effect is ascribed in various indications.

Infection-relevant pathogens exist, such as Staphylococcus aureus, Staphylococcus epidermidis, Streptococcus pyogenes, Streptococcus pneumoniae, or Haemophilus influenzae. Among them is also a Staphylococcus aureas strain that is resistant to methicillin, called MRSA. Standard antibiotics such as beta-lactam antibiotics, for example oxacillin, penicillin and amoxicillin, increasingly no longer have an effect on this bacterium, because they have developed resistances through the excessive use of antibiotics, which do not fully destroy the pathogenic agents. Such bacteria represent an additional risk in the case of skin-mucosa penetration, which could result in lung inflammations, wound infections, and blood poisoning or other life-threatening infections.

In the prior art, WO 2004/064533 describes that produced plant hydrolysates having enzyme effect can be produced and can be used in cosmetics. German Patent Specification DE 35 39 492 describes a method for hydrolysis of pulverized lignocellulose using concentrated hydrochloric acid and subsequent distillation reclamation of the hydrochloric acid used and re-hydrolysis of the sugar solution formed after dilution with water.

It is the object of the present invention to provide a cosmetic or dermatological composition or an agent for body care and beauty care, which unfolds a skin-care and skin-protecting action.

Surprisingly, cosmetic or dermatological compositions containing hydrolysates of extracts of at least one plant drugs from the respective genera, in particular the species Althaeae radix (marshmallow root), Angelica(e) dahurica (dahurian angelica), Angelica(e) sinensis (Chinese angelica), Armoracia rusticana (horseradish), Artemisia scoparia (redstem wormwood), Astragalus membranaceus (var. mongolicus) (Chinese milk vetch), Capsicum sp., Capsicum annuum (pepper), Centaurium erythraea (common centaury), Cistus incanus (hairy rockrose), Echinacea angustifolia (narrow-leaved purple coneflower), Echinacea purpurea (Eastern purple coneflower), Equiseti herba (horsetail), Galphimia glauca, Gentiana lutea L. (great yellow gentian), Guaiacum officinalis, Hederae folium, Hedera helicis (ivy), Juglandis folium (walnut leaf), Leonurus japonicus (Chinese motherwort), Levesticum radix, Levisticum officinalis (lovage), Matricariae flos (or Flos chamomillae (chamomile flower)), Melia toosendan (chinaberry fruit), Millefolii herba (yarrow), Olea europaea (olive), Pelargonium sp. (geranium), Phytolacca americana (American pokeweed), Primulae flos, Primulae radix, Primula veris L. (cowslip), Quercus cortex (oak bark), Rosmarinus officinalis (rosemary), Rumicis herba (sorrel herb), Salix sp. (willow), Salvia miltiorrhiza (red sage), Sambucus nigra L. (black elder), Saposhnikovia divaricata (siler), Scutellaria baicalensis (Baikal skullcap), Siegesbeckia pubescens (Siegesbeckia), Taraxaci herba (dandelion root herb), Thymus L. (thyme), Verbena officinalis L. (vervain), Vitex agnus castus (monk's pepper), Vitis vinifera (common grapevine), display an effective antibacterial effect.

This is particularly advantageous for skin protection.

The invention relates in particular to a cosmetic or dermatological composition containing a hydrolysate made of at least one extract, which is produced by extraction from dried plant material from:

a.) at least one of the plants selected from the group comprising:
the respective genera, in particular the species Althaeae radix (marshmallow root), Angelica(e) dahurica (dahurian angelica), Angelica(e) sinensis (Chinese angelica), Armoracia rusticana (horseradish), Artemisia scoparia (redstem wormwood), Astragalus membranaceus (var. mongolicus) (Chinese milk vetch), Capsicum sp., Capsicum annuum (pepper), Centaurium erythraea (common centaury), Cistus incanus (hairy rockrose), Echinacea angustifolia (narrow-leaved purple coneflower), Echinacea purpurea (Eastern purple coneflower), Equiseti herba (horsetail), Galphimia glauca, Gentiana lutea L. (great yellow gentian), Guaiacum officinalis, Hederae folium, Hedera helicis (ivy), Juglandis folium (walnut leaf), Leonurus japonicus (Chinese motherwort), Levesticum radix, Levisticum officinalis (lovage), Matricariae flos (or Flos chamomillae (chamomile flower)), Melia toosendan (chinaberry fruit), Millefolii herba (yarrow), Olea europaea (olive), Pelargonium sp. (geranium), Phytolacca americana (American pokeweed), Primulae flos, Primulae radix, Primula veris L. (cowslip), Quercus cortex (oak bark), Rosmarinus officinalis (rosemary), Rumicis herba (sorrel herb), Salix sp. (willow), Salvia miltiorrhiza (red sage), Sambucus nigra L. (black elder), Saposhnikovia divaricata (siler), Scutellaria baicalensis (Baikal skullcap), Siegesbeckia pubescens (Siegesbeckia), Taraxaci herba (dandelion root herb), Thymus L. (thyme), Verbena officinalis L. (vervain), Vitex agnus castus (monk's pepper), Vitis vinifera (common grapevine); and a mixture or subcombination thereof,
   subsequently removing the extraction agent, the hydrolysate being obtainable from the extract by hydrolytic treatment using a mineral acid.

Therefore, the invention also relates to a cosmetic or dermatological composition for topical use containing a hydrolysate made of an extract, which is produced by extraction from dried plant material, the hydrolysate being obtainable from the extract by hydrolytic treatment using a mineral acid.

The plants (plant drugs) according to the invention can be obtained, as is customary for the respective plant drug, from preferred parts of the plants, such as leaves, roots and the like.

A preferred hydrolyzate is characterized in that the extracts can be produced from the plant material by means of an extracting agent comprising 40 to 60% by volume, in particular 50% by volume ethanol and 40 to 60% by volume, in particular 50% by volume water over 24 hours while stirring and subsequent vacuum evaporation of the solvent.

A further preferred embodiment of the invention is a hydrolyzate which can be obtained by the hydrolytic treatment of the plant extracts using hydrochloric acid as the mineral acid, in particular using hydrochloric acid having a concentration of 1 M to 10 M, preferably 6 to 9 M, in particular approximately 8 M, at 80° C. to 100° C., in particular approximately 90° C., for 30 minutes to 120 minutes, in particular 40 minutes to 60 minutes, preferably approximately 45 minutes. In the final solution, the concentration of the hydrochloric acid is preferably 1 to 4 M, in particular 1 to 2 M, in particular 1.3 M.

It is preferred to carry out the hydrolytic treatment of the extracts in the presence of ethanol, in particular ethanol diluted with water, preferably 50% by volume ethanol.

Within the context of this invention, the term “hydrolyzate” denotes an aqueous phase obtained from the extract of the plant (plant drug) according to the invention, in which the hydrolysis products are enriched. The hydrolysis preferably takes place under the action of acid, such as hydrochloric acid, phosphoric acid, sulfuric acid, mineral acid, in particular diluted mineral acid. The extract can be obtained, for example, by means of an aqueous-ethanolic extraction from a plant (plant drug), admixed by means of aqueous acid, evaporated to dryness, and subsequently received in water. The hydrolysis causes the chemical cleavage of ingredients, wherein formally hydrogen and hydroxide are added to the respective cleavage product. The hydrolysis brings about a change of the substance composition of the hydrolyzate as compared to the existing known aqueous/ethanolic extracts.

In order to ensure that the hydrolyzates of the present invention have good physiological compatibility, the extracts are evaporated to dryness after the acid treatment step, received preferably in water, a buffer, or in diluted ethanol, and optionally neutralized with a pharmaceutically acceptable base. Possible bases are, for example, NaOH, Na₂CO₃ or Na₂HPO₄, without being exhaustive.

It came as a complete surprise when it was found that the cosmetic or dermatological compositions according to the invention, which contain hydrolyzates, have an antibacterial effect.

In a preferred embodiment of a cosmetic or dermatological composition, the content of hydrolysates according to the invention is 0.1 to 30% by weight, preferably 0.5 to 10% by weight, or preferably 1.0 to 5% by weight.

The hydrolyzates of the present invention generally have a significant antibacterial effect, which in the therapeutic range thereof is comparable to an antibiotic control agent comprising amoxicillin and clavulanic acid (mass ratio of 6:1). Therefore, a skin-protecting function benefits these hydrolysates. Furthermore, the hydrolysates display a curative and palliative effect on the skin flora.

In particular, skin diseases, in particular bacterially caused skin diseases, can be treated by means of the hydrolysates of the present invention, such as skin eczema, lip herpes, acne (vulgaris, inter alia), and skin infections of all types. For example, the hydrolysates display effectiveness against Propionibacterium acne and is therefore particularly suitable for the treatment and prophylaxis of acne.

The hydrolysates were tested and found to be effective against the following pathogens within the context of the present invention:

Staphylococcus aureus (ATCC 25923), Staphylococcus epidermidis (ATCC 12228), Streptococcus pneumoniae (DSMZ 20566), Streptococcus pyogenes (DSMZ 20565), Streptococcus mutans (ATCC 35668), Haemophilus influenzae (DSMZ 4690), Klebsiella pneumoniae (ATCC 13883), and Enterococcus casseliflavus (VRE) (DSMZ 20680) as well as against the intestinal bacteria Escherichia coli (ATCC 25922), Enterococcus faecalis (VRE) (ATCC 19433), and Pseudonomas aeruginosa.

In an advantageous way which is known per se, the hydrolysates of the present invention can be used to produce a cosmetic or dermatological composition or an agent for body care and beauty care.

Therefore, the invention also relates to a pharmaceutical consisting of a dermatological composition containing hydrolysates of the present invention for the prophylaxis and treatment of skin diseases, such as skin eczema, lip herpes, acne (vulgaris, inter alia), and skin infections of all types.

In a further embodiment, the cosmetic or dermatological preparation according to the invention can be produced for topical use in the form of a salve, cream, gel, lotion, paste, or preferably an emulsion. Water-free systems are also possible.

Emulsions are understood in general as heterogeneous systems, which consist of two liquids which are non-miscible or only miscible to a limited extent with one another, which are typically referred to as phases. In an emulsion, one of the two liquids is dispersed in the form of ultrafine droplets in the other liquid. If the two liquids are water and oil and oil droplets are provided finely distributed in water, this is an oil-in-water emulsion (O/W emulsion). The basic character of an O/W emulsion is characterized by the water. In the case of a water-in-oil emulsion (W/O emulsion), the reverse principle is involved, in which the basic character is determined here by the oil. Furthermore, mixed systems such as water-in-oil-in-water emulsions (W/O/W emulsions) and oil-in-water-in-oil emulsions (O/W/O emulsions) are known. All mentioned emulsions are suitable according to the invention.

The water-free systems according to the invention include pure oil preparations such as skin oils. Pastes, which are also usable, containing the preparation according to the invention are distinguished in that they consist of the same or similar components as an emulsion but are substantially water-free. Within the context of the present invention, the terms oil phase and lipid phase are used synonymously. In a further preferred embodiment, the preparation according to the invention can contain an emulsifier as a further component. In a very preferred embodiment, this emulsifier can be an O/W emulsifier.

Emulsifiers can advantageously be selected from the group of non-ionic, anionic, cationic, or amphoteric emulsifiers.

Various emulsifiers from the group of partial fatty acid esters, fatty alcohols, sterols, polyethylene glycols, such as ethoxylated fatty acids, ethoxylated fatty alcohols, and ethoxylated sorbitan esters, sugar emulsifiers, polyglycerol emulsifiers, or silicone emulsifiers can be used as non-ionic emulsifiers.

Various emulsifiers from the group of soaps, e.g., sodium stearate, fatty alcohol sulfates, monoalkyl, dialkyl, and trialkyl phosphoric acid esters and the ethoxylates thereof, fatty acid lactate esters, fatty acid citrate esters, or fatty acids citroglycerol esters can be used as anionic emulsifiers.

For example, quaternary ammonium compounds having a long chain aliphatic residue, such as distearyl dimonium chloride, can be used as cationic emulsifiers.

Various emulsifiers from the groups alkylamino alkane carboxylic acids, betaines, sulfobetaines, or imidazole derivatives can be used as amphoteric emulsifiers.

According to the invention, naturally occurring emulsifiers, which include, for example, beeswax, lanolin, lecithin, and sterols, inter alia, for example, may preferably be used in the production of a preparation according to the invention. In a preferred formulation of the preparation according to the invention, O/W emulsifiers may be selected from the group of plant protein hydrolysates and the derivatives thereof.

Furthermore, advantageous substances can be included as additives as defined in the present invention, selected from the group of esters of saturated and/or unsaturated, branched and/or unbranched alkane carboxylic acids and/or alkene carboxylic acids having a chain length of 3-30 carbon atoms and saturated and/or unsaturated, branched and/or unbranched alcohols of a chain length of 3-30 carbon atoms and from the group of esters of aromatic carboxylic acids and saturated and/or unsaturated, branched and/or unbranched alcohols of a chain length of 3-30 carbon atoms. Such esterols can advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl iso-nonanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semisynthetic, and natural mixtures of such esters, such as jojoba oil.

Furthermore, the oil phase can advantageously be selected from the group of branched and unbranched hydrocarbons and waxes, the dialkyl ethers, the group of saturated or unsaturated, branched or unbranched alcohols, and the fatty acid triglycerides, namely the triglycerol esters of saturated and/or unsaturated, branched and/or unbranched alkane carboxylic acids of a chain length of 8-24 carbon atoms, in particular 12-18 carbon atoms. The fatty acid triglycerides can advantageously be selected from the group of synthetic, semisynthetic, and natural oils, for example.

Therefore, the invention also relates to a cosmetic or dermatological composition according to the invention, which contains, for example, jojoba oil, olive oil, sunflower oil, soybean oil, peanut oil, canola oil, almond oil, palm oil, coconut oil, palm kernel oil, or the like as additives. Vegetable oils which have at least 6% by weight polyunsaturated fatty acids are very particularly preferred.

In a further preferred embodiment, the cosmetic or dermatological compositions according to the invention contain at least 1% by weight, preferably 1 to 30% by weight, particularly preferably 2 to 15% by weight or even 5 to 10% by weight of at least one such vegetable oil as an additive.

In particular, antioxidants and/or radical scavengers can additionally be added to the preparations according to the invention as auxiliary agents or additives. Such antioxidants are advantageously selected from the group of lipophilic systems, for example: natural and synthetic tocopherols, nordihydroguaiaretic acid, coniferyl benzoate, butylhydroxy anisole, butylhydroxy toluene, gallic acid ester, and various antioxidant plant extracts. Among the hydrophilic systems, inorganic sulfur compounds, sodium hydrogen sulfite, cysteine, or ascorbic acid are particularly advantageously to be used.

The cosmetic or dermatological compositions according to the invention may further contain cosmetic auxiliary agents, as are typically used in such preparations, such as preservatives, bactericidal agents, perfumes, anti-foaming substances, colorants, pigments, which have a coloring effect (e.g., for decorative cosmetics), thickeners, surfactants, softening, moistening, and/or moisture-retaining substances, or other typical components of a cosmetic or dermatological formulation, such as alcohols, polyols, polymers, foam stabilizers, electrolytes, organic solvents, or silicone derivatives.

Furthermore, a component of coenzyme Q10, hyaluronic acid, collagen, paracress, aloe vera, jojoba, and/or at least one vitamin. Furthermore, optionally typical anti-aging active ingredients, peeling agents, corrosion inhibitors, antioxidants, antistatic agents, and/or at least one conditioner, binder, and/or skin lightener.

In a further specific embodiment, the preparation according to the invention is substantially composed of naturally occurring ingredients.

In microbiological studies at the Institute for Hygiene at the Medical University of Innsbruck, it has surprisingly been shown that the hydrolysates according to the invention have a broad, sometimes pronounced antibacterial effect against skin-relevant pathogens, which was significantly more pronounced in its antibacterial effect in corresponding tests than was the case with non-hydrolyzed extracts. Thus, for example, in antibacterial sensitivity tests using the agar diffusion test according to Mueller-Hinton (Mueller, H. J. and Hinton, J. (1941): A protein-free medium for primary isolation of the Gonococcus and Meningococcus. Proc. Soc. Expt. Biol. Med.; 48:330-333), it was shown that of the hydrolyzed individual drug extracts, the hydrolysates according to the invention were effective against multiple pathogens and the majority of non-hydrolyzed mixtures surprisingly displayed practically no antibacterial effect against the tested bacteria reference panel in the agar diffusion test.

In addition, it is essential that this respective achieved antibacterial effectiveness improve a palliative and curative effect of the plant (drugs) according to the invention, also in a respective combination.

In a further embodiment, the invention relates to a method for producing hydrolysates, obtainable from at least one aqueous/ethanolic extract from at least one plant (plant drug) according to the invention, an aqueous/ethanolic extract preferably being admixed with aqueous acid and subsequently the soluble fractions being collected (=hydrolysate).

The invention also relates to a method for producing a dermatological agent having antibacterial effect, a preferably aqueous/ethanolic extract being produced from at least one plant (plant drug) according to the invention in a first step, and the obtained extract being admixed with an aqueous acid and the aqueous fractions being collected and optionally dried in a second step.

In a further embodiment of the invention, the obtained hydrolysates can be converted into a dry compound. In a preferred embodiment, freeze-drying of the hydrolysates is performed. Other drying methods are also usable, however.

Both the hydrolysates according to the invention and also the aqueous suspensions and dried compounds thereof display an antibacterial effect and therefore an advantageous skin-protecting effect.

Reference is made to the technical teaching which is the subject matter of EP 1368605B1 and EP 0753306B1 on the production of the extracts according to the invention and the combinations thereof made of the plants.

Further advantages and features of the present invention result on the basis of the description of exemplary embodiments. The following examples serve to explain the invention without restricting the invention to these examples.

### EXAMPLES

### Preparation of the Test Solutions

The individual drugs and mixed extracts having variable drug compositions were extracted in 50% EtOH/H₂O (v/v, approximately 1 g plant material for 20 ml solvent) for 24 hours at room temperature while stirring. 1.6 ml extract were admixed with 320 μl 25% HCl (corresponding to 8.1 mol/l) and 80 n150% EtOH and hydrolyzed for 45 minutes at 90° C. For comparison, in a second step, the hydrolysis was conducted with 1 ml extract under the same conditions, while adding 1 ml 25% HCl. After concentrating the extracts by evaporation, the residue was received in 1 ml sterile water and tested for the antibacterial effect thereof. Screening method: 80 μl of the test solution were placed on Mueller Hinton agar plates, or Mueller Hinton Agar plates with 5% sheep blood, which contained an unknown concentration of the bacteria to be tested, and incubated for 24 hours at 37° C.

Spiral platter (SP): A bacteria colony was suspended in 5 ml CASO-Bouillon and incubated for 24 hours at 37° C. The supernatant was removed after centrifuging the sample, washed with 0.9% NaCl, and diluted to a concentration of 10⁷ cfu/ml (colony-forming units per milliliter). The test solutions were diluted 1:2, 1:20, and 1:200 and mixed with the bacteria suspension (for Pneumococcus and H. influenzae: 1:10, for the remaining pathogens 1:100). 0.9% NaCl was used for positive control purposes. The samples were plated with a Whitley Automatic Spiral Platter (WASP) after 0, 4, and 8 hours and incubated for 24 hours at 37° C.

Tables: Results of Antimicrobial Effect:

Screening tests: Individual drugs/mixed extract before hydrolysis

TABLE 1
Results of the assay for antibacterial effects of the respective individual extracts

| | | | | | | 5 Pfl. |
|---|---|---|---|---|---|---|
| | Gentiana l. | Sambucus n. | Verbena o. | Rumex h. | Primula v. | Extrakt |
| Staph. aureus | Ø | Ø | Ø | + | +M | Ø |
| P. aeruginosa | Ø | +B | Ø | +B | Ø | Ø |
| Pneumococcus | Ø | Ø | Ø | +M | +M | Ø |
| Strept. pyogenes | Ø | Ø | Ø | +M | +M | Ø |
| Klebsiella | Ø | Ø | Ø | Ø | Ø | Ø |
| E. coli | Ø | Ø | Ø | Ø | Ø | Ø |
| H. influenzae | Ø | Ø | Ø | Ø | Ø | Ø |
| Staph. epidermidis | Ø | Ø | Ø | + | Ø | Ø |
| Ent. faecalis (VRE) | Ø | Ø | Ø | + | Ø | Ø |
| Ent. casseliflavus | Ø | Ø | Ø | +M | Ø | Ø |
| (VRE) | | | | | | |

Gentiana lutea (Gentiana l.), Sambucus nigra (Sambucus n.), Verbena officinalis (Verbena o.), Rumex herba (Rumex h.) and Primula veris (Primula v.) and a mixed extract of all five plants (5 Pfl. Extrakt = 5 plant extract) against the pathogenic agents Staphylococcus aureus (Staph. aureus), Pseudomonas aeruginosa (P. aeruginosa), Pneumococcus, Streptococcus pyogenes (Strept. pyogenes), Klebsiella, Escherichia coli (E. coli), Haemophilus influenzae (H. influenzae), Staphylococcus epidermidis (Staph. epidermidis), Enterococcus faecalis (VRE) (Ent. faecalis (VRE)), and Enterococcus casseliflavus (VRE) (Ent. casseliflavus (VRE)).
+M = antibacterial activity on Mueller Hinton agar;
+B = antibacterial activity on Mueller Hinton agar with 5% sheep blood;
+ = effect on both plates

TABLE 2
Quantification of the antimicrobial effect of Rumex
herba and of Primula veris by means of spiral plating
against the pathogens mentioned in Table 1.

| | |
|---|---|
| Rumex h. | Primula v. |

| | 1:20 | 1-200 | 1:20 | 1-200 |
|---|---|---|---|---|
| Staph. aureus | +++ | + | ++ | (+) |
| P. aeruginosa | Ø | Ø | Ø | Ø |
| Pneumococcus | ++++ | ++++ | ++++ | ++++ |
| Strept. pyogenes | ++++ | ++++ | ++++ | ++ |
| Klebsiella | Ø | Ø | Ø | Ø |
| E. coli | Ø | Ø | Ø | Ø |
| H. influenzae | Ø | Ø | Ø | Ø |
| Staph. epidermidis | ++ | ++ | Ø | Ø |
| Ent. faecalis (VRE) | ++++ | ++++ | Ø | Ø |
| Ent. caselliflavus (VRE) | ++++ | ++++ | Ø | Ø |

The samples were each quantified in 1:20 or 1:200 dilutions.
++++ = 10² cfu/ml after 0 hours,
+++ = 10² cfu/ml after 4 hours,
++ = 10² cfu/ml after 8 hours,
+ = 10³-10⁴ cfu/ml after 8 hours,
(+) = higher activity in comparison to the control group
Screening tests: Plant mixtures in various compositions, non-hydrolyzed

TABLE 3
50% ethanolic mixed extracts consisting of respectively 5, 4, or 3 plants were produced
from the individual drugs mentioned in Table 1 and assayed for their antimicrobial
effect against the pathogenic agents mentioned in Table 1 in screening tests.

| | RGVSP | RVSP | RGVP | RGSP | RGVS | GVSP | RVP | VSP | GVP | GVS | RSP |
|---|---|---|---|---|---|---|---|---|---|---|---|
| Staph. aureus | Ø | + | + | + | (+) | (+) | Ø | Ø | Ø | Ø | (+) |
| P. aeruginosa | Ø | Ø | Ø | Ø | Ø | Ø | Ø | Ø | Ø | Ø | Ø |
| Pneumococcus | Ø | Ø | Ø | Ø | Ø | Ø | (+) | Ø | Ø | Ø | Ø |
| Strept. pyogenes | Ø | Ø | Ø | Ø | Ø | Ø | Ø | Ø | Ø | Ø | + |
| Klebsiella | Ø | Ø | Ø | Ø | Ø | Ø | Ø | Ø | Ø | Ø | Ø |
| E. coli | Ø | Ø | Ø | Ø | Ø | Ø | Ø | Ø | Ø | Ø | Ø |
| H. influenzae | Ø | Ø | Ø | Ø | Ø | Ø | Ø | Ø | Ø | Ø | Ø |
| Staph. epidermidis | Ø | Ø | Ø | Ø | Ø | Ø | Ø | Ø | Ø | Ø | Ø |
| Ent. faecalis (VRE) | Ø | Ø | Ø | Ø | Ø | Ø | Ø | Ø | Ø | Ø | Ø |
| Ent. Casseliflavus | Ø | Ø | Ø | Ø | Ø | Ø | Ø | Ø | Ø | Ø | Ø |
| (VRE) | | | | | | | | | | | |

| | RGP | RGS | RGV | RVS | GSP |
|---|---|---|---|---|---|
| Staph. aureus | + | Ø | Ø | Ø | Ø |
| P. aeruginosa | Ø | Ø | Ø | Ø | Ø |
| Pneumococcus | Ø | Ø | Ø | Ø | Ø |
| Strept. pyogenes | Ø | + | Ø | Ø | Ø |
| Klebsiella | Ø | Ø | Ø | Ø | Ø |
| E. coli | Ø | Ø | Ø | Ø | Ø |
| H. influenzae | Ø | Ø | Ø | Ø | Ø |
| Staph. epidermidis | Ø | Ø | Ø | Ø | Ø |
| Ent. faecalis (VRE | Ø | Ø | Ø | Ø | Ø |
| Ent. casseliflavus | Ø | Ø | Ø | Ø | Ø |
| (VRE) | | | | | |

Gentiana lutea (G), Sambucus nigra (S), Verbena officinalis (V), Rumex herba (R), and Primula veris (P).
+ = effect on both plates (Muller Hinton agar, Muller Hinton agar with 5% sheep blood),
(+) = effect on one plate

TABLE 4
The obtained extracts of the plant drugs mentioned in Table 1 and a mixed extract
consisting of all five plants were hydrolyzed by means of hydrochloric acid and tested
against the pathogenic agents mentioned in Table 1 for antibacterial effect.

| | | | | | | 5 Pfl. |
|---|---|---|---|---|---|---|
| | Gentiana l. | Sambucus n. | Verbena o. | Rumex h. | Primula v. | Extrakt |
| Staph. aureus | + | + | Ø | Ø | + | + |
| P. aeruginosa | + | (+) | Ø | Ø | + | + |
| Pneumococcus | + | + | + | Ø | + | + |
| Strept. pyogenes | + | + | + | Ø | + | + |
| Klebsiella | + | (+) | Ø | Ø | + | + |
| E. coli | + | Ø | Ø | Ø | + | + |
| H. influenzae | + | + | Ø | Ø | + | + |
| Staph. epidermidis | + | + | + | Ø | + | + |
| Ent. faecalis (VRE) | + | Ø | Ø | Ø | + | + |
| Ent. casseliflavus | + | Ø | Ø | Ø | + | + |
| (VRE) | | | | | | |

The extracts were concentrated by vaporization until dry after completed hydrolysis and dissolved in sterile water.
+M = antibacterial activity on Mueller Hinton agar;
+ = effect on both plates (Muller Hinton agar, Muller Hinton agar with 5% sheep blood),
(+) = effect on one plate

TABLE 5
Quantification of the obtained hydrolysates of the plant drugs
mentioned in Table 1 and of the 5-plant extract by means of
spiral plating against the pathogens mentioned in Table 1.

| | Gentiana | Sambucus | Verbana | Primula | 5 Pfl. |
|---|---|---|---|---|---|
| | l. | n. | o. | v. | Extrakt |
| Staph. aureus | (+) | (+) | + | +++ | (+) |
| P. aeruginosa | ++++ | +++ | ++++ | ++++ | ++++ |
| Pneumococcus | +++ | + | (+) | ++++ | |
| Strept. pyogenes | ++++ | ++++ | ++++ | ++++ | ++++ |
| Klebsiella | +++ | ++ | ++ | +++ | +++ |
| E. coli | +++ | (+) | (+) | + | + |
| H. influenzae | ++++ | ++++ | ++++ | ++++ | ++++ |
| Staph. | +++ | +++ | +++ | ++++ | +++ |
| epidermidis | | | | | |
| Ent. | +++ | ++ | + | +++ | +++ |
| faecalis | | | | | |
| (VRE) | | | | | |
| Ent. | +++ | ++ | +++ | +++ | +++ |
| caselliflavus | | | | | |
| (VRE) | | | | | |

All solutions were measured in 1:20 dilution.
++++ = 10² cfu/ml after 0 hours,
+++ = 10² cfu/ml after 4 hours,
++ = 10² cfu/ml after 8 hours,
+ = 10³-10⁴ cfu/ml after 8 hours,
(+) = higher activity in comparison to the control group
Comparison of the reproducibility of the antibacterial effect

TABLE 6
Study of the reproducibility of the antibacterial effectiveness of multiple extracts of
the same individual drugs (Sambucus nigra, Gentiana lutea, Primula veris).

| Sambucus n. | Extrakt 1 | Extrakt 2 | Extrakt 3 |
|---|---|---|---|
| Staph. aureus | (+) | (+) | (+) |
| P. aeruginosa | +++ | +++ | ++++ |
| Pneumococcus | + | ++ | |
| Strept. Pyogenes | ++++ | +++ | +++ |
| Klebsiella | ++ | (+) | +++ |
| E. coli | (+) | (+) | |
| H. influenzae | ++++ | ++++ | |
| Staph. epidermidis | +++ | ++ | ++++ |
| Ent. faecalis (VRE) | ++ | (+) | ++ |
| Ent. caselliflavus (VRE) | ++ | ++ | +++ |
| Gentiana l. | Extrakt 1 | Extrakt 2 | Extrakt 3 |
| Staph. aureus | (+) | (+) | (+) |
| P. aeruginosa | ++++ | ++++ | ++++ |
| Pneumococcus | +++ | ++++ | |
| Strept. pyogenes | ++++ | ++++ | ++++ |
| Klebsiella | +++ | +++ | ++++ |
| E. coli | +++ | + | ++++ |
| H. influenzae | ++++ | ++++ | |
| Staph. epidermidis | +++ | +++ | ++++ |
| Ent. faecalis (VRE) | +++ | ++ | +++ |
| Ent. caselliflavus (VRE) | +++ | +++ | ++++ |

| Primula v. | Extrakt 1 | Extrakt 2 | Extrakt 3 | Extrakt 4 | Extrakt 5 |
|---|---|---|---|---|---|
| Staph. aureus | +++ | (+) | ++ | (+) | + |
| P. aeruginosa | ++++ | +++ | ++++ | ++++ | ++++ |
| Pneumococcus | ++++ | (+) | +++ | +++ | +++ |
| Strept. pyogenes | ++++ | +++ | ++++ | ++++ | +++ |
| Klebsiella | +++ | (+) | ++++ | + | +++ |
| E. coli | + | (+) | +++ | ++ | + |
| H. influenzae | ++++ | +++ | ++++ | ++++ | ++++ |
| Staph. epidermidis | ++++ | (+) | +++ | +++ | +++ |
| Ent. faecalis | +++ | (+) | +++ | + | + |
| (VRE) | | | | | |
| Ent. | +++ | ++ | +++ | +++ | ++ |
| caselliflavus | | | | | |
| (VRE) | | | | | |

Quantification by means of spiral plating in 1:20 dilution of the sample.
++++ = 10² cfu/ml after 0 hours,
+++ = 10² cfu/ml after 4 hours,
++ = 10² cfu/ml after 8 hours,
+ = 10³-10⁴ cfu/ml after 8 hours,
(+) = higher activity in comparison to the control group
Extrakt = extract

TABLE 7
Tested extracts

| | | Equiseti | | Juglandis | | Millefolii | | Quercus | | Taraxaci | | Althaeae | | Matricariae | |
|---|---|---|---|---|---|---|---|---|---|---|---|---|---|---|---|
| | | Platte | SP | Platte | SP | Platte | SP | Platte | SP | Platte | SP | Platte | SP | Platte | SP |
| 1 | Staph. aureus | Ø | | (+) | | Ø | | + | | Ø | | Ø | | Ø | |
| 2 | P. aeroginosa | Ø | | (+) | | Ø | | + | | Ø | | Ø | | Ø | |
| 3 | Pneumococcus | + | | Ø | | Ø | | Ø | | Ø | | Ø | | Ø | |
| 4 | Streptococcus pyogenes | Ø | | + | | Ø | | + | | Ø | | Ø | | Ø | |
| 5 | Klebsiella | Ø | | + | | Ø | | + | | Ø | | Ø | | Ø | |
| 6 | E. coli | Ø | | Ø | | Ø | | Ø | | Ø | | Ø | | Ø | |
| 7 | H. influenzae | Ø | | Ø | | Ø | | (+) | | Ø | | Ø | | Ø | |
| 8 | Staph. epidermidis | Ø | | + | | Ø | | + | | Ø | | Ø | | Ø | |
| 9 | Ent. faecalis (VRE) | Ø | | Ø | | Ø | | (+) | | Ø | | Ø | | Ø | |
| 10 | Ent. casilliflavus | Ø | | Ø | | Ø | | Ø | | Ø | | Ø | | Ø | |
| | (VRE) | | | | | | | | | | | | | | |

| | | Althaeae | | Equiseti | | Taraxaci | | Quercus | | Matricariae | | Millefolli | | Junglandis | |
|---|---|---|---|---|---|---|---|---|---|---|---|---|---|---|---|
| | | hydrolysiert | | hydrolysiert | | hydrolysiert | | hydrolysiert | | hydrolysiert | | hydrolysiert | | hydrolysiert | |
| | | Platte | SP | Platte | SP | Platte | SP | Platte | SP | Platte | SP | Platte | SP | Platte | SP |
| 1 | Staph. aureus | Ø | | (+) | | Ø | | + | | Ø | | (+) | | Ø | |
| 2 | P. aeroginosa | Ø | | (+) | | Ø | | + | | (+) | | (+) | | Ø | |
| 3 | Pneumococcus | + | | + | | Ø | | (+) | | + | | + | | + | |
| 4 | Streptcoccus pyogenes | Ø | | Ø | | Ø | | + | | (+) | | (+) | | Ø | |
| 5 | Klebsiella | Ø | | (+) | | Ø | | (+) | | Ø | | Ø | | Ø | |
| 6 | E. coli | Ø | | (+) | | Ø | | (+) | | Ø | | Ø | | Ø | |
| 7 | H. influenzae | + | | (+) | | Ø | | + | | (+) | | (+) | | Ø | |
| 8 | Staph. epidermidis | Ø | | (+) | | Ø | | + | | Ø | | (+) | | Ø | |
| 9 | Ent. faecalis (VRE) | Ø | | (+) | | Ø | | + | | Ø | | (+) | | Ø | |
| 10 | Ent. casilliflavus | Ø | | (+) | | Ø | | (+) | | (+) | | Ø | | Ø | |
| | (VRE) | | | | | | | | | | | | | | |

+ effect on both plates;
(+) effect on one plate;
Ø no effect

TABLE 8
Composition Canephron:
Results of the antibacterial activity

| | Centaurium | | Levisticum | | Rosmarinus | |
|---|---|---|---|---|---|---|
| | erythraea | | officianale | | officinalis | |
| | no | | no | | no | |
| Bacterial strains | hydrol. | hydrol. | hydrol. | hydrol. | hydrol. | hydrol. |
| Staphylococcus aureus | Ø | + | Ø | + | + | + |
| Pseudomonas aeruginosa | Ø | + | Ø | + | Ø | + |
| Streptococcus pneumonia | Ø | + | Ø | + | (+) | + |
| Streptococcus pyogenes | Ø | + | Ø | + | + | + |
| Klebsiella pneumoniae | Ø | + | Ø | + | (+) | (+) |
| Eschericia coli | Ø | (+) | Ø | + | Ø | Ø |
| Haemophilus influenzae | Ø | (+) | Ø | + | Ø | + |
| Staphylococcus epidermidis | Ø | Ø | Ø | + | + | + |
| Enterococcus faecalis (VRE) | Ø | Ø | Ø | (+) | + | + |
| Enterococcus casseliflavus | Ø | (+) | Ø | + | + | + |
| (VRE) | | | | | | |

Analysis:
“+” = antibacterial activity;
“(+)” = slight antibacterial activity,
“Ø” = no activity

TABLE 9
Results of the antibacterial activity

| | Angelica | | Angelica | | Artemisia | |
|---|---|---|---|---|---|---|
| | dahurica | | sinensis | | scoparia | |
| | no | | no | | no | |
| Bacterial strains | hydrol. | hydrol. | hydrol. | hydrol. | hydrol. | hydrol. |
| Staphylococcus aureus | Ø | + | Ø | + | Ø | Ø |
| Pseudomonas aeruginosa | Ø | + | Ø | + | Ø | + |
| Streptococcus pneumonia | Ø | + | + | + | Ø | (+) |
| Streptococcus pyogenes | Ø | + | Ø | + | Ø | (+) |
| Klebsiella pneumoniae | Ø | + | Ø | + | Ø | Ø |
| Escherichia coli | Ø | + | Ø | + | Ø | Ø |
| Haemophilus influenzae | Ø | + | Ø | + | Ø | + |
| Staphylococcus epidermidis | Ø | + | Ø | + | Ø | Ø |
| Enterococcus faecalis (VRE) | Ø | + | Ø | (+) | Ø | Ø |
| Enterococcus casseliflavus | Ø | + | Ø | + | Ø | Ø |
| (VRE) | | | | | | |
| | Saposhnikovia | | Scutellaria | | Siegesbeckia | |
| | divaricata | | baicalensis | | pubescens | |
| | no | | no | | no | |
| Bacterial strains | hydrol. | hydrol. | hydrol. | hydrol. | hydrol. | hydrol. |
| Staphylococcus aureus | Ø | + | Ø | + | Ø | (+) |
| Pseudomonas aeruginosa | Ø | + | Ø | Ø | Ø | + |
| Streptococcus pneumonia | Ø | + | Ø | + | Ø | + |
| Streptococcus pyogenes | Ø | + | Ø | (+) | Ø | + |
| Klebsiella pneumoniae | Ø | + | Ø | (+) | Ø | + |
| Escherichia coli | Ø | + | Ø | (+) | Ø | (+) |
| Haemophilus influenzae | Ø | + | Ø | + | Ø | (+) |
| Staphylococcus epidermidis | Ø | + | Ø | Ø | Ø | (+) |
| Enterococcus faecalis (VRE) | Ø | + | Ø | (+) | Ø | (+) |
| Enterococcus casseliflavus | Ø | + | Ø | Ø | Ø | (+) |
| (VRE) | | | | | | |

Analysis:
“+” = antibacterial activity;
“(+)” = slight antibacterial activity,
“Ø” = no activity

TABLE 10

| | Results of the antibacterial activity | | | |
|---|---|---|---|---|
| | Armoracia rusticana | | Capsicum sp. | |
| | no | | no | |
| Bacterial strains | hydrol. | hydrol. | hydrol. | hydrol. |
| Staphylococcus aureus | Ø | + | Ø | + |
| Pseudomonas aeruginosa | Ø | + | Ø | + |
| Streptococcus pneumonia | Ø | + | + | + |
| Streptococcus pyogenes | Ø | + | Ø | + |
| Klebsiella pneumoniae | Ø | + | Ø | + |
| Escherichia coli | Ø | + | Ø | + |
| Haemophilus influenzae | Ø | + | Ø | + |
| Staphylococcus epidermidis | Ø | + | Ø | + |
| Enterococcus faecalis (VRE) | Ø | + | Ø | + |
| Enterococcus casseliflavus | Ø | + | Ø | + |
| (VRE) | | | | |

| | Results of the antibacterial activity | | | | | |
|---|---|---|---|---|---|---|
| | | | Ech. purpurea | | Ech. purpurea | |
| | Cistus incanus | | radix | | herba | |
| | no | | no | | no | |
| Bacterial strains | hydrol. | hydrol. | hydrol. | hydrol. | hydrol. | hydrol. |
| Staphylococcus aureus | + | + | Ø | + | Ø | (+) |
| Pseudomonas aeruginosa | Ø | + | Ø | + | Ø | + |
| Streptococcus pneumonia | (+) | + | Ø | + | Ø | + |
| Streptococcus pyogenes | (+) | + | Ø | + | Ø | + |
| Klebsiella pneumoniae | Ø | + | Ø | + | Ø | + |
| Escherichia coli | Ø | + | Ø | + | Ø | (+) |
| Haemophilus influenzae | Ø | + | Ø | + | Ø | (+) |
| Staphylococcus epidermidis | + | + | Ø | + | Ø | Ø |
| Enterococcus faecalis (VRE) | + | + | Ø | + | Ø | + |
| Enterococcus casseliflavus | Ø | + | Ø | + | Ø | Ø |
| (VRE) | | | | | | |
| | Results of the antibacterial activity | | | | | |
| | Galphimia | | | | Melia | |
| | glauca | | Hedera helicis | | toosendan | |
| | no | | no | | no | |
| Bacterial strains | hydrol. | hydrol. | hydrol. | hydrol. | hydrol. | hydrol. |
| Staphylococcus aureus | (+) | (+) | Ø | (+) | Ø | + |
| Pseudomonas aeruginosa | Ø | Ø | Ø | + | Ø | + |
| Streptococcus pneumonia | Ø | + | (+) | + | + | + |
| Streptococcus pyogenes | (+) | + | + | (+) | Ø | + |
| Klebsiella pneumoniae | Ø | Ø | Ø | + | Ø | + |
| Escherichia coli | Ø | Ø | Ø | (+) | Ø | + |
| Haemophilus influenzae | Ø | (+) | + | (+) | Ø | + |
| Staphylococcus epidermidis | (+) | (+) | (+) | Ø | Ø | + |
| Enterococcus faecalis (VRE) | Ø | Ø | Ø | Ø | Ø | + |
| Enterococcus casseliflavus | Ø | + | Ø | + | Ø | + |
| (VRE) | | | | | | |
| | Results of the antibacterial activity | | | | | |
| | | | Pelargonium | | Phytolacca | |
| | Olea europaea | | sp. | | Americana | |
| | no | | no | | no | |
| Bacterial strains | hydrol. | hydrol. | hydrol. | hydrol. | hydrol. | hydrol |
| Staphylococcus aureus | + | + | + | + | Ø | + |
| Pseudomonas aeruginosa | Ø | + | Ø | + | Ø | + |
| Streptococcus pneumonia | Ø | + | Ø | + | Ø | Ø |
| Streptococcus pyogenes | Ø | + | + | + | Ø | Ø |
| Klebsiella pneumoniae | (+) | + | Ø | + | Ø | + |
| Escherichia coli | Ø | + | Ø | + | Ø | + |
| Haemophilus influenzae | Ø | + | Ø | + | Ø | Ø |
| Staphylococcus epidermidis | + | + | + | + | Ø | + |
| Enterococcus faecalis (VRE) | Ø | + | + | + | Ø | + |
| Enterococcus casseliflavus | Ø | + | + | + | Ø | Ø |
| (VRE) | | | | | | |
| | Results of the antibacterial activity | | | | | |
| | Primula versis | | | | | |
| | radix | | Salix sp. | | Thymus L. | |
| | no | | no | | no | |
| Bacterial strains | hydrol. | hydrol. | hydrol. | hydrol. | hydrol. | hydrol. |
| Staphylococcus aureus | + | + | + | + | + | + |
| Pseudomonas aeruginosa | Ø | + | + | + | Ø | + |
| Streptococcus pneumonia | + | + | + | + | Ø | + |
| Streptococcus pyogenes | + | + | + | + | + | + |
| Klebsiella pneumoniae | Ø | + | + | + | Ø | + |
| Escherichia coli | Ø | + | + | + | Ø | + |
| Haemophilus influenzae | (+) | + | + | (+) | Ø | (+) |
| Staphylococcus epidermirdis | (+) | Ø | + | + | Ø | + |
| Enterococcus faecalis (VRE) | (+) | (+) | + | + | Ø | + |
| Enterococcus casseliflavus | Ø | (+) | + | + | Ø | + |
| (VRE) | | | | | | |
| | Results of the antibacterial activity | | | | | |
| | Vitex agnus | | Vitis | | Ech. | |
| | castus | | vinifera | | Augustifolia | |
| | no | | no | | no | |
| Bacterial strains | hydrol. | hydrol. | hydrol. | hydrol. | hydrol. | hydrol. |
| Staphylococcus aureus | Ø | + | + | + | Ø | + |
| Pseudomonas aeruginosa | Ø | + | Ø | + | Ø | + |
| Streptococcus pneumonia | Ø | + | Ø | + | Ø | + |
| Streptococcus pyogenes | (+) | + | Ø | + | Ø | + |
| Klebsiella pneumoniae | Ø | + | Ø | + | Ø | + |
| Escherichia coli | Ø | (+) | Ø | + | Ø | + |
| Haemophilus influenzae | Ø | + | Ø | + | Ø | + |
| Staphylococcus epidermidis | Ø | (+) | Ø | + | Ø | + |
| Enterococcus faecalis (VRE) | Ø | + | Ø | + | Ø | + |
| Enterococcos casseliflavus | Ø | (+) | Ø | + | Ø | + |
| (VRE) | | | | | | |
| | Plate diffusion test | | | | | |
| | Huang qi | | Ch. motherwort | | Red sage | |
| | no | | no | | no | |
| Bacterial strains | hydrol. | hydrol. | hydrol. | hydrol. | hydrol. | hydrol. |
| Staphylococcus aureus | Ø | + | Ø | + | + | + |
| Pseudomonas aeruginosa | Ø | + | Ø | + | Ø | + |
| Streptococcus pneumoniae | Ø | + | (+) | + | + | + |
| Streptococcus pyogenes | Ø | + | + | + | (+) | + |
| Klebsiella pneumoniae | Ø | + | (+) | + | Ø | + |
| Escherichia coli | Ø | + | Ø | + | Ø | + |
| Haemophilus influenzae | Ø | + | Ø | + | Ø | + |
| Staphylococcus epidermidis | Ø | + | Ø | + | + | + |
| Enterococcus faecalis (VRE) | Ø | + | Ø | + | + | + |
| Enterococcus casseliflavus | Ø | + | Ø | + | + | + |
| (VRE) | | | | | | |

Analysis:
“+” = antibacterial activity;
“(+)” = slight antibacterial activity,
“Ø” = no activity
Ech. = Echinacea
Legend: Huang qi (Astragalus membranaceus), Chinese motherwort (Leonurus japonicus), Red sage (Salvia miltiorrhiza).

The MIC of the cultivated bacteria is determined according to DIN58940-1. The minimal inhibiting concentration, MIC in short, is the smallest active ingredient concentration of an antimicrobial substance (e.g., an antibiotic), which still prevents the pathogen reproduction in the culture. MIC 90: minimal inhibiting concentration for 90% of the tested strains. A statement about the effectiveness of an antibiotic or the resistance of a specific pathogen may be made on the basis of the minimal inhibiting concentration. The determination is performed by various methods of antibiotic resistance testing, e.g., by the agar dilution test, the plate diffusion test, or an E test and is typically specified in μg/ml.

The agar dilution test was applied: 10 mL of a brain heart infusion (BHI) or Fildes broth (for H. Influenza) was inoculated with five colonies of the bacteria strains and subcultivated for 18-22 hours at 36+/−1° C. with 50 or 100 μL it of a standardized bacteria suspension in the culture for 2.5 hours at 36+/−1° C. Subsequently, the bacteria cultures were centrifuged and resuspended in sodium phosphate buffer (10 mM, pH 7.2-7.4) until reaching a total concentration of 10⁵ colonies (colony forming units/milliliters or cfu/ml). Immediately after standardization of the growing inoculum, 100 μL of the bacteria test strain was added to the extract test solution in 96-well microtitration plates. Positive growth controls of bacteria strains in the extract test solutions were performed using basal medium.

Determination of the endpoints: MIC 90 values were determined by counting the colonies according to DIN 59049-7, two hours after incubation, with slight modifications, as follows: 100 μL, of a bacteria culture were admixed in a test tube with 1 μL of a sterile 0.9% sodium chloride solution, followed by a tenfold dilution in each case, 10¹, 10², and 10³. 100 μL was then removed and painted twice on an agar plate, an action time of 18-24 hours at 36+/−1° C. in the presence of room air or CO₂ for Streptococcus sp. occurring. The cfu/ml (or mg/ml) were then ascertained and extrapolated. All tests were performed twice simultaneously and the arithmetic mean was determined

### Results:

| | |
|---|---|
| | Primulae |
| MIC₉₀ | radix |

| in [mg/ml] | non | |
|---|---|---|
| Gram-positive | hydrol. | hydrol. |
| Staph. aureus | 0.025 | 6.25 |
| Staph. aureus | 0.2 | 3.125 |
| (MRSA¹) | | |
| Staph. | 0.2 | 3.125 |
| epidermidis | | |
| Strep. pyogenes | 0.025 | 3.125 |
| Strep. | 0.025 | 3.125 |
| pneumoniae | | |
| Strep. mutans | * | 3.125 |
| Ent. faecalis | 0.1 | 6.25 |
| (VRE²) | | |

MRSA¹: multiresistant Staphylococcus aureus;
VRE²: vancomycin resistant enterococcus
n.d.: Not determined;
* >25 mg/mL

| | MIC₉₀ in [mg/ml] | | | | |
|---|---|---|---|---|---|
| | | | | Levistici | |
| | Primulae flos | | Primul. | radix | |
| | non | | radix | non | |
| Gram-negative | hydrol. | hydrol. | hydrol. | hydrol. | hydrol. |
| Escherichia coli | * | 12.5 | 3.125 | * | 6.25 |
| Pseudomonas | * | 12.5 | 3.125 | * | 6.25 |
| aeruginosa | | | | | |
| Haemoph. | 3.125 | 6.25 | 3.125 | 0.2 | 6.25 |
| influenzae | | | | | |
| Haemoph. | 1.56 | 3.125 | 3.125 | 0.2 | 1.56 |
| influenzae | | | | | |
| Klebsiella | 1.56 | 12.5 | 3.125 | * | 6.25 |
| pneumoniae | | | | | |
| Klebsiella | 6.25 | 12.5 | 3.125 | * | 6.25 |
| pneumoniae | | | | | |
| (ESBL³) | | | | | |
| Burkholderia | 12.5 | 12.5 | 3.125 | * | 6.25 |
| cepacia | | | | | |

ESBL³: extended spectrum beta lactamase
n.d.: Not determined;
* >25 mg/mL

Guaiacum

| | no | |
|---|---|---|
| Bakterienstämme | hydrol. | hydrol. |
| Staph. aureus | + | + |
| Ps. aeruginosa | (+) | Ø |
| Strep. pyogenes | + | + |
| Strep. pneumoniae | (+) | (+) |
| K. pneumoniae | Ø | Ø |
| E. coli | + | + |
| Haemoph. influenzae | + | + |

Bakterienstämme = bacterial strains

1. A cosmetic or dermatological composition for topical use containing a hydrolysate of an extract, which is produced by extraction from dried plant material, wherein the hydrolysate is obtainable from the extract by hydrolytic treatment with a mineral acid.

2. A cosmetic or dermatological composition for topical use containing a hydrolysate of at least one extract, which is produced by extraction from dried plant material, made of:
a.) at least one of the plants selected from the group consisting of at least one genus: Althaeae, Angelica(e), Armoracia, Artemisia, Astragalus, Capsicum, Centaurium, Cistus, Echinacea, Equiseti, Galphimia, Gentiana, Guaiacum, Hedera, Juglandis, Leonurus, Levisticum, Matricariae, Melia, Millefolii, Olea, Pelargonium, Phytolacca, Primula, Quercus, Rosmarinus, Rumicis, Salix, Salvia, Sambucus, Saposhnikovia, Scutellaria, Siegesbeckia, Taraxaci, Thymus, Verbena, Vitex, Vitis; and a mixture or subcombination thereof; or
b.) at least one of the plants selected from the group consisting of at least one species: Althaeae radix (marshmallow root), Angelica(e) dahurica (dahurian angelica), Angelica(e) sinensis (Chinese angelica), Armoracia rusticana (horseradish), Artemisia scoparia (redstem wormwood), Astragalus membranaceus (var. mongolicus) (Chinese milk vetch), Capsicum sp., Capsicum annuum (pepper), Centaurium erythraea (common centaury), Cistus incanus (hairy rockrose), Echinacea angustifolia (narrow-leaved purple coneflower), Echinacea purpurea (Eastern purple coneflower), Equiseti herba (horsetail), Galphimia glauca, Gentiana lutea L. (great yellow gentian), Guaiacum officinalis, Hederae folium, Hedera helicis (ivy), Juglandis folium (walnut leaf), Leonurus japonicus (Chinese motherwort), Levesticum radix, Levisticum officinalis (lovage), Matricariae flos (or Flos chamomillae (chamomile flower)), Melia toosendan (chinaberry fruit), Millefolii herba (yarrow), Olea europaea (olive), Pelargonium sp. (geranium), Phytolacca americana (American pokeweed), Primulae flos, Primulae radix, Primula veris L. (cowslip), Quercus cortex (oak bark), Rosmarinus officinalis (rosemary), Rumicis herba (sorrel herb), Salix sp. (willow), Salvia miltiorrhiza (red sage), Sambucus nigra L. (black elder), Saposhnikovia divaricata (siler), Scutellaria baicalensis (Baikal skullcap), Siegesbeckia pubescens (Siegesbeckia), Taraxaci herba (dandelion root herb), Thymus L. (thyme), Verbena officinalis L. (vervain), Vitex agnus castus (monk's pepper), Vitis vinifera (common grapevine), and a mixture or subcombination thereof, subsequent removal of the extraction agent,
characterized in that the hydrolysate is obtainable from the extract by hydrolytic treatment with a mineral acid.

3. The cosmetic or dermatological composition for topical use containing a hydrolysate according to claim 1, wherein the extracts are producible by means of an extraction agent made of 40 to 60% by volume ethanol and 40 to 60% by volume water from the plant material over 6 to 36 hours, with stirring and optional vacuum concentration by evaporation of the solvent.

4. The cosmetic or dermatological composition for topical use containing a hydrolysate according to claim 1, wherein it is obtainable by hydrolytic treatment of the extracts with hydrochloric acid as the mineral acid.

5. The cosmetic or dermatological composition for topical use containing hydrolysate according to claim 1, wherein the hydrolysate has an antibacterial effect.

6. The cosmetic or dermatological composition for topical use containing hydrolysate according to claim 1, wherein the content is 0.1-30% by weight.

7. The cosmetic or dermatological composition for topical use according to claim 1, in the form of a cream, lotion, paste, gel, skin oil, or emulsion.

8. The cosmetic or dermatological composition for topical use according to claim 1, characterized in that it contains at least one further vegetable oil as an additive, which contains at least 6% by weight polyunsaturated fatty acids.

9. An agent for body care and beauty care consisting of a cosmetic or dermatological composition according to claim 1.

10. A pharmaceutical consisting of a dermatological composition according to claim 1 for the prophylaxis and treatment of skin diseases, such as skin eczema, lip herpes, acne, and skin infections.

11. The cosmetic or dermatological composition for topical use containing a hydrolysate according to claim 1, wherein the extracts are producible by means of an extraction agent made of 40 to 60% by volume ethanol and 40 to 60% by volume water from the plant material over 12 to 30 hours, with stirring and optional vacuum concentration by evaporation of the solvent.

12. A cosmetic or dermatological composition for topical use containing a hydrolysate according to claim 1, wherein it is obtainable by hydrolytic treatment of the extracts with hydrochloric acid as the mineral acid, in a concentration of 1 M to 10 M, at 80 to 100° C., for 30 to 120 min., and/or the final solution has a concentration of the hydrochloric acid of 1 to 4 M.

13. A cosmetic or dermatological composition for topical use containing a hydrolysate according to claim 1, wherein it is obtainable by hydrolytic treatment of the extracts with hydrochloric acid as the mineral acid, in a concentration of 6 M to 9 M, at 80 to 100° C., for 40 to 60 min. and/or the final solution has a concentration of the hydrochloric acid of 1 to 2 M.
